(19) United States
(12) Patent Application Publication
(10) Pub. No.: US 2024/0066088 A1
(43) Pub. Date: Feb. 29, 2024

(54) INFUSED NASAL STRIP

(71) Applicant: UAB Orbio World, Kaunas (LT)

(72) Inventor: Vitalija Kreiviene, Kaunas (LT)

(73) Assignee: UAB Orbio World, Kaunas (LT)

(21) Appl. No.: 17/900,798

(22) Filed: Aug. 31, 2022

(51) Int. Cl. A61K 36/53; A61F 5/08; A61L 31/04; A61L 31/16

(52) U.S. Cl. CPC A61K 36/53; A61F 5/08; A61L 31/041; A61L 31/16; A61L 2300/30

(57) ABSTRACT

The present invention relates to a natural nasal strip with the strip arranged to provide congestion relief and increased airflow to provide calming and relaxing benefits to the body. The nasal strip provides the traditional benefits of typical nasal strips with a composition of components forming the strip that minimize irritation. The components include, but are not limited to a spun lace, non-woven fabric, polyester comprised of diol and dicarboxylic acid, nonallergenic adhesive, and silicone paper. Additionally, the composition may include an optional lavender oil in the nonallergenic adhesive.

### BACKGROUND OF THE INVENTION

### 1. Field of the Invention

The present invention relates to the modification of a nasal strip device. More specifically, the present invention relates to a nasal strip device which is infused with ingredients to assist in breathing as well as improve body functions and health. Still more specifically, the present invention is a nasal strip a non-woven fabric layer, an elastic strip layer, and a silicone paper layer designed to help promote sleep health.

### 2. Description of the Prior Art

Humans have developed a variety of methods, medicines, and other treatments for various ailments. These treatments may be of a physical nature, chemical nature, mental nature, or come in another form. Treatments are typically designed to address certain ailments. The best treatment may be dependent on the individual's response to the treatment and the type of ailment. One such ailment is nasal congestion. One such treatment for nasal congestion is the use of nasal strips.

Traditional nasal strips are used to open the nasal passage to facilitate easier breathing. Current nasal strips are adhered to each side of the nose, extending over the bridge of the nose. The nasal strip is designed to utilize physics to widen the nasal passage. The current technology provides a method to assist breathing during sleep or conditions in which a user is experiencing limited oxygen intake through their nose.

In addition to physical treatments such as the use of nasal strips, treatments for certain ailments may include introducing the body to natural materials. The body may take in these materials by ingesting, inhaling, absorbing, or other utilizing other methods to provide the body with access to the desired materials. The current technology to provide the body access with these materials is through ingestion of the materials in some form (pill form, direct nutrient intake, or other methods of ingestion). Current technology fails to provide a natural approach for providing the body with a method to absorb the desired nutrients without the need for ingestion, inhalation, or other methods.

Further, current technology fails to provide a method which treats congestion with a physical method but also provides the body with detoxification capabilities. What is needed is a nasal strip which assists a user with breathing and congestion relief that also provides the body with ingredients to promote detoxification. What is needed is a nasal strip device that can accomplish the traditional uses for a nasal strip while also providing additional health benefits. The nasal strip should provide the body with additional nutrients and health benefits. What is needed is a nasal strip that also infuses the body with beneficial nutrients and ingredients to assist with additional bodily functions.

### SUMMARY OF THE INVENTION

The present invention is a nasal strip infused with ingredients to allow the body to absorb ingredients and nutrients while using the nasal strip. The nasal strip may be effective for individuals who are focused on health and fitness but not limited to that. The nasal strip is designed to facilitate easier breathing.

It is a goal of the present invention to provide a nasal strip that provides traditional benefits of aiding in breathing but also to provide additional health benefits to the user passively. The present invention contains three layers including a layer of spun lace, non-woven fabric, a layer of elastic material, and a layer of silicone paper. Additionally, the present invention has an adhesive to adhere the device to the nose. The spun lace, non-woven fabric layer is formed of a cotton and/or synthetic fiber material. The elastic layer is formed of a high molecular compound made by condensation polymerization of equal amount of diol and dicarboxylic acid. The third layer is a removable paper layer, which is removed before use, to expose the adhesive used to removably affix the strip to the exterior surface of the nose. The adhesive is a pressure-sensitive adhesive material. Additionally, the adhesive may be infused with a lavender oil. The present invention provides a mechanical benefit to opening the nostrils of the user with minimal adhesive contact. It optionally provides a further benefit of supplying lavender oil adjacent to the nose. These benefits include but are not limited to improved sleep and energy, decreased fatigue, and may result in improved cognitive performance.

The present invention provides the mechanical benefit of opening the nasal passage to increase airflow by relieving blockages or congestion. The present invention utilizes natural ingredients to achieve the desired function with minimal chance of skin irritation that may occur with other similar products. It also provides an optional soothing lavender scent through the infusion of lavender oil.

The present invention adheres to each side of the outside of the nose, spanning across the bridge of the nose. The present invention has a flexible middle which draws the two locations of adherence together to pull open the nasal passage. Additionally, the present invention is infused with natural ingredients and nutrients, which, when in contact with the skin, provide calming and relaxing benefits to the body.

### BRIEF DESCRIPTION OF THE DRAWINGS

FIG. 1 is a top perspective view of an embodiment of the infused nasal strip.

FIG. 2 is a cross-sectional view of an embodiment of the present invention.

FIG. 3 is a top perspective view of the present invention with perforated portion.

FIG. 4 is a top perspective view of the present invention.

### DETAILED DESCRIPTION OF THE INVENTION

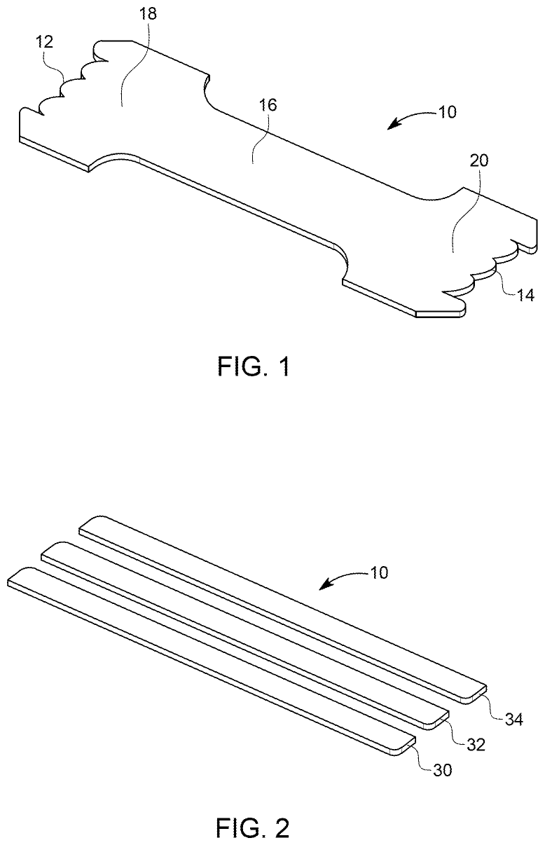

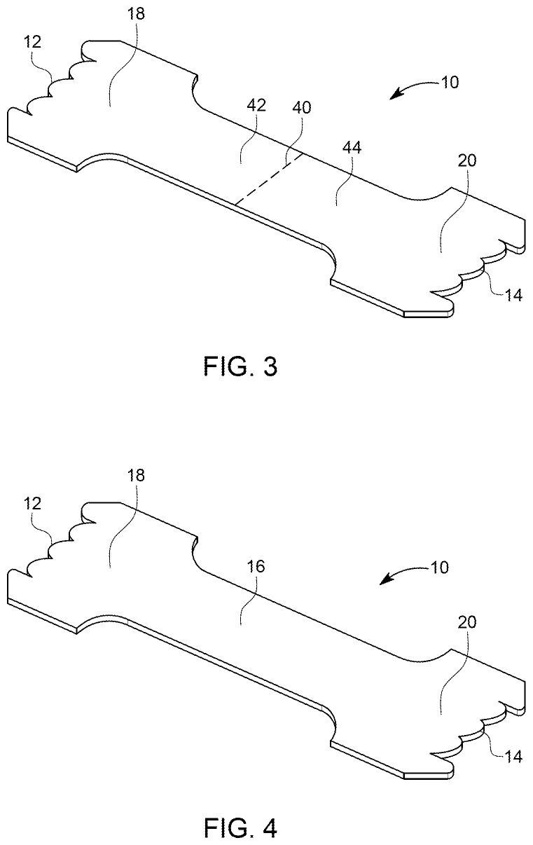

An infused nasal strip 10 of the present invention is shown in FIGS. 1-4. The nasal strip 10 has a first end 12, a second end 14, and a main body 16. The nasal strip 10 has a first layer 30, a second layer 32, and a third layer 34. The first end 12 has a first adhesion site 18 and the second end 14 has a second adhesion site 20. The first adhesion site 18 and the second adhesion site 20 are located on the second layer 32. The main body 16 may also have a main body adhesion site 21. The third layer 34 is a removable cover 40. The removable cover 40 may be made up of a single, removable piece, or multiple removable pieces, designed to expose the adhesion site 18 and adhesion site 20 of the second layer 32.

When put to use, the first end 12 and the second end 14 are positioned on each side of the bridge of the nose. The first adhesion site 18 secures the first end 12 of the nasal strip 10 to one side of the nose and the second adhesion site 20 secures the second end 14 of the nasal strip 10 to the other side of the nose. The main body 16 is aligned between the first end 12 and the second end 14. The first adhesion site 18 and the second adhesion site 20 of the second layer 32 may extend between the first end 12 and the second end 14, respectively, to the main body 16 such that the nasal strip 10 effectively has continuous adhesion throughout the length of the device 10 on the second layer 32. Alternatively, the first adhesion site 18 and the second adhesion site 20 may be located on the first end 12 and the second end 14, respectively, with the main body 16 having no adhesion characteristic.

FIG. 2 depicts a cross-sectional view of the nasal strip first layer 30, the second layer 32, and the third layer 34. The first layer 30 is made of a material providing flexible structure and comfort to the nasal strip 10. The first layer 30 may be made of a spun lace non-woven fabric. The second layer 32 is an elastic strip with adhesive properties. The second layer 32 may be made of a polyester and a pressure sensitive adhesive. The polyester of the second layer 32 may be a high molecular compound formed by condensation polymerization of equal amount of diol and dicarboxylic acid, with elasticity. The third layer 34 is a removable cover 40. The removable cover 40 may be a silicone paper.

The second layer 32 has a thin layer of adhesive material that is preferably hypoallergenic but does not have to be. The second layer 32 is made of an antiallergic adhesive. It is selected to ensure that the nasal strip 10 remains in place on the location of interest while also being removable when the infusion activity is completed. The removable cover 40 has a first second 42 and a second section 44. The removable cover 40 and the second layer 32 perform much like the release paper and adhesive surface of a bandage. The second layer 32 includes adhesive material located on or within the first adhesion site 18 and second adhesion site 20, and optionally, on or within the main body 16. The first layer 30 is formed of a non-metallic material having features that provide comfortable benefits to the body by enhancing the chance of getting a good sleep with minimal chance of skin irritation.

The third layer 34 may cover a portion or the entirety of the second layer 32 and first layer 30. The third layer 34 may be integrated with the second layer 32. It is arranged to maintain the nasal strip 10 in a stored manner while retaining the natural materials of the composition therein until such a time as the nasal strip 10 is applied to the skin. The third layer 34 is fabricated of a non-metallic material such as a spun lace non-woven fabric but not limited thereto. The third layer 34 is made of a flexible material so that the nasal strip 10 may be formed to the desired location, which may include noses of variable size and shape.

In a first embodiment, the present invention includes spun lace non-woven fabric, polyester comprised of diol and dicarboxylic acid, silicone paper, and an antiallergic adhesive. The polyester composition may be about 50% diol and about 50% dicarboxylic acid. The antiallergic adhesive is comprised of a resin and granular rubber. The antiallergic adhesive may also contain an optional lavender oil.

When in use, the first section 42 and the second section 44 of the removable cover 40 are removed from the second layer 32 to expose the first adhesion site 18 and the second adhesion site 20. The first adhesion site 18 and the second adhesion site 20 are applied to the desirable location of the nose, such that the first end 12 and the second end 14 of the nasal strip 10 are located on either side of the nose with the connector 16 extending over the bridge of the nose. The nasal strip 10 in its entirety is pressed firmly to the nose to ensure the second layer 32 has as much contact with the skin as possible. The nasal strip 10 is then left in place for a selectable period of time, such as the time while sleeping but not limited thereto.

What is claimed is:

1. A natural treatment nasal strip for removable application to skin, the nasal strip comprising:
a first layer comprising a spun lace non-woven fabric;
a second layer comprising a polyester;
a third layer comprising a silicone paper; and
an adhesive for removably joining the nasal strip body to the skin; and
wherein the polyester includes a diol and dicarboxylic acid.

2. The nasal strip of claim 1 wherein the nasal strip includes lavender oil.

3. The adhesive of claim 1 wherein the adhesive is comprised of resin and granular rubber.

4. The adhesive of claim 1 wherein the adhesive is comprised of resin, granular rubber, and the lavender oil is about 0.01% to about 0.05% by weight of the composition.
